(19) United States
(12) Patent Application Publication
(10) Pub. No.: US 2018/0296507 A1
(43) Pub. Date: Oct. 18, 2018

(54) REDOX-ACTIVATED GLYCOCONJUGATED PROCHELATORS

(71) Applicant: ARIZONA BOARD OF REGENTS ON BEHALF OF THE UNIVERSITY OF ARIZONA, Tucson, AZ (US)

(72) Inventors: Elisa Tomat, Tucson, AZ (US); Eman A. Akam, Tucson, AZ (US)

(21) Appl. No.: 16/014,905

(22) Filed: Jun. 21, 2018

Related U.S. Application Data
(63) Continuation-in-part of application No. PCT/US16/68061, filed on Dec. 21, 2016.
Application No. PCT/US16/68061 is a continuation-in-part of application No. 15/345,393, filed on Nov. 7, 2016.
Application No. 15/345,393 is a continuation of application No. 14/531,634, filed on Nov. 3, 2014, now Pat. No. 9,486,423.
(60) Provisional application No. 62/270,246, filed on Dec. 21, 2015.
(60) Provisional application No. 61/899,262, filed on Nov. 3, 2013.

(51) Int. Cl. A61K 31/175

(52) U.S. Cl. CPC A61K 31/175

(57) ABSTRACT

Compositions of iron prochelator compounds conjugated to a carbohydrate moiety are described herein. The glycoconjugates are prochelators that are activated in reducing conditions, such as in the intracellular space, so as to sequester iron. The glycoconjugate molecules are taken up by cells via the glucose transporter and may be used to target malignant cells.

### CROSS REFERENCE

This application is a continuation-in-part and claims benefit of PCT Application No. PCT/US16/68061, filed Dec. 21, 2016, which claims benefit of U.S. Provisional Application No. 62/270,246, filed Dec. 21, 2015, the specification(s) of which is/are incorporated herein in their entirety by reference.

This application is a continuation-in-part and claims benefit of U.S. patent application Ser. No. 15/345,393, filed Nov. 7, 2016, which is a continuation and claims benefit of U.S. patent application Ser. No. 14/531,634, filed Nov. 3, 2014, now U.S. Pat. No. 9,486,423, which claims benefit of U.S. Provisional Patent Application No. 61/899,262, filed Nov. 3, 2013, the specification(s) of which is/are incorporated herein in their entirety by reference.

### FIELD OF THE INVENTION

The present invention relates to chelators such as iron chelators, more particularly, to redox-activated glycoconjugated prochelators. In some embodiments, the present invention relates to modified iron prochelators linked to a carbohydrate moiety.

### BACKGROUND OF THE INVENTION

Cancer cells are believed to require higher iron levels in order to sustain fast proliferation rates. For example, cell walls in various tumor types are characterized by the upregulation of the transferrin receptor, TfR1, which is responsible for increased cellular uptake of the iron transport protein transferrin (Tf). In addition, cancer cells have high glucose demands for their sustained growth. This aspect of malignant behavior may be targeted by the use of small-molecule scavengers (e.g., chelators) that interfere with the availability of intracellular iron. Indeed, iron chelators used for iron overload treatment (e.g., the siderophore desferrioxamine (DFO)) and thiosemicarbazones, such as Triapine, have been used therapeutically to attempt to reduce proliferation of cancer cells (see examples of iron chelators in FIG. 2). However, chelation therapies in cancer treatment lack the ability to target iron ions in malignant cells without affecting iron levels in the bloodstream and in normal tissue. Their applicability has been hampered by dose-limiting toxicity and reported occurrence of adverse side effects, including, but not limited to, hearing abnormalities, renal toxicity, optic neuropathy, and growth failure in children). To date, no iron chelator has been approved for clinical use in cancer chemotherapy.

The present invention features novel compounds that can concurrently exploit two physiological characteristics of malignant cells: (1) their marked glucose avidity; and (2) their susceptibility to iron deprivation. By selectively depriving cancer cells of iron, a metal ion essential for rapid proliferation, the novel compounds could disrupt cancer cell metabolism selectively over normal tissue and extracellular space, while reducing the side effects as compared to existing iron chelators.

Inventors have previously reported redox-activated iron prochelators (see U.S. Pat. No. 9,486,423, and U.S. Provisional Application No. 61/899,262). For example, compound (TC1-S)₂ shown below comprises two thiosemicarbazone-based molecules linked by a disulfide bond (a thiosemicarbazone dimer), which masks the sulfur atoms in the S,N,S donor sets, rendering the chelator a prochelator; the disulfide bond linking the thiosemicarbazone molecules significantly reduces or eliminates chelation of iron in a relatively non-reducing environment (e.g., blood plasma).

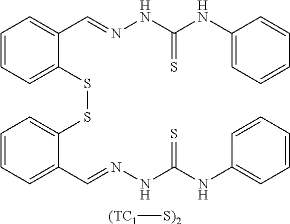

A reducing environment (e.g., the intracellular space of a cell such as a cancer cell) triggers the activation of the chelator via cleavage of the disulfide bond. The resulting chelator TC1-SH (see below) readily sequesters iron. Such prochelators were shown to exhibit antiproliferative activity in several cell lines.

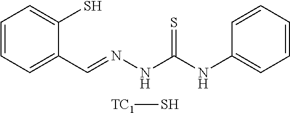

The present invention features glycoconjugated prochelators that may be used to reduce or eliminate proliferation of cells, e.g., cancer cells. As such, the present invention also features methods of reducing or eliminating proliferation of a cell and methods of treating conditions associated with iron dysregulation (e.g., methods of treating a cancer) by treating the cells or patients with glycoconjugated prochelators of the present invention. The present invention further features methods of synthesis of glycoconjugated prochelators.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### SUMMARY OF THE INVENTION

According to one embodiment, the present invention features prochelators comprising an iron prochelator conjugated with a carbohydrate moiety (or a derivative thereof). In some embodiments, the iron prochelator may comprise a disulfide bond and at least two donor atoms. In some embodiments, the iron prochelator and the carbohydrate moiety are linked by a linker comprising the disulfide bond.

In some embodiments, the carbohydrate moiety comprises glucose molecule or a mannose molecule or another sugar moiety. In some embodiments, the carbohydrate moiety is linked at its C2 carbon to the iron prochelator. In other embodiments, the carbohydrate moiety is linked at its C6 carbon or at a different position, e.g., a position other than the C2 or C6 position of the carbohydrate moiety. The carbohydrate moiety can allow the prochelator compound to be taken up by a cell via a glucose transporter protein, such as GLUT1.

In some embodiments, the iron prochelator is susceptible to activation by reduction. For example, the iron prochelator is susceptible to activation by a redox reaction. In other embodiments, the iron prochelator is activated upon entry into intracellular space of a cell to form an iron chelator that inhibits proliferation of a cell via sequestration of iron by the iron prochelator.

According to another embodiment, the present invention also features methods of inhibiting proliferation of a cancer cell. The method may comprise introducing to the cell a glycoconjugated prochelator as described herein. According to a further embodiment, the present invention features a composition for use in inhibiting proliferation of a cancer cell. The composition may comprise a glycoconjugated prochelator compound as described herein, in a pharmaceutically acceptable carrier.

In some embodiments, the present invention also features methods of treating a condition associated with a metal ion dysregulation. The method may comprise administering to a subject a therapeutically effective amount of a prochelator compound as described herein. In other embodiments, the present invention also features a composition for use in treating a condition associated with a metal ion dysregulation. The composition may comprise a glycoconjugated prochelator compound as described herein, in a pharmaceutically acceptable carrier.

The present invention also features methods of synthesizing a glycoconjugated prochelator compound according to the present invention. In some embodiments, the method may comprise initiating amidic or ester coupling of pyridyl disulfide crosslinker with a carbohydrate moiety (or derivative thereof) to produce a cross-linked carbohydrate, and initiating disulfide exchange of the cross-linked carbohydrate with an iron chelator to produce the glycoconjugated prochelator compound. In one embodiment, the pyridyl disulfide crosslinker is 3-(2-pyridyldithio)propionic acid. Examples of the iron chelator include, but are not limited to, a thiosemicarbazone, semicarbazone, aroylhydrazone, desferrioxamine (DFO), deferiprone (DFP), hydroxypyridinone, deferasirox (DFX), desferrithiocin (DFT), or analogs thereof.

In other aspects, the present invention additionally features methods of increasing water solubility of an iron prochelator. In some embodiments, the method comprises conjugating a carbohydrate moiety (or a derivative thereof) to the iron chelator, thereby producing a glycoconjugated prochelator (e.g., a glycoconjugated prochelator according to any described herein). In preferred embodiments, the carbohydrate moiety can increase the water solubility of the prochelator.

One of the unique and inventive technical features of the present invention is that the iron prochelators that can be conjugated with a carbohydrate moiety (or a derivative thereof) using a disulfide linkage of the prochelator. The glycoconjugate prochelators can be activated in reducing conditions so as to transform into chelators that sequester iron. Without wishing to limit the invention to any theory or mechanism, the novel glycoconjugate molecules are taken up by cells via the glucose transporter since they compete with 2-(N-(7-Nitrobenz-2-oxa-1,3-diazol-4-yl)amino)-2-deoxyglucose (2-NBDG) for cell entry. As indicated by the data that will be presented herein, due to the increased expression of glucose transporters in the malignant cells, the glycoconjugated prochelators demonstrate higher toxicity toward malignant cells, instead of normal cells, and cell uptake of the glycoconjugated prochelator in the malignant cells is enhanced. This technical feature of the present invention advantageously provides for glycoconjugated iron prochelators that preferentially deprive cancer cells of iron.

Further still, the glycoconjugate prochelators can have increased water solubility as compared to the unconjugated iron prochelator. Thus, the glycoconjugate prochelators can have an enhanced bioavailability. None of the presently known prior references or work has the unique inventive technical feature of the present invention.

### BRIEF DESCRIPTION OF THE DRAWINGS

The features and advantages of the present invention will become apparent from a consideration of the following detailed description presented in connection with the accompanying drawings in which:

FIG. 1A and 1B show exemplary mechanisms of a redox-directed glycoconjugate prochelator strategy. This novel prochelation strategy utilizes a disulfide redox switch to connect an iron-binding unit with a carbohydrate moiety to target increased expression of glucose transporters in cancer cells. Reduction of the glycoconjugates in the intracellular space results in the generation of a thiolate and formation of a low-spin Fe(III) complex.

FIG. 2 shows non-limiting examples of chelators that sequester iron in biological settings (desferrioxamine (DFO), deferiprone (DFP), deferasirox (DFX), triapine, and Dp44mT).

FIG. 3 shows disulfide-containing thiosemicarbazones (thiosemicarbazone dimers) (e.g., (TC1S)₂, (TC2S)₂, (TC3S)₂) that behave as low-affinity prochelators, releasing high-affinity chelators (e.g., TC1SH, TC2SH, TC3SH) upon reduction of the disulfide bond.

FIG. 4 shows resulting iron complexes based on X-ray crystallographic data (note BF₄⁻ counter ion not shown). These chelators stabilize iron centers as low-spin Fe(III) species.

FIG. 5 shows a non-limiting schematic for preparing compounds through condensation of 2,2′-dithiodibenzaldehyde or diketone precursors with appropriate thiosemicarbazide, semicarbazides, or hydrazides.

FIG. 6 shows a synthesis scheme for glycoconjugated prochelators.

FIG. 7 shows an alternative synthesis scheme for glycoconjugated prochelators.

FIGS. 8A-8D show exemplary mechanisms for synthesizing sugar analogues into glycoconjugated prochelators (FIGS. 8A-8C), and for synthesizing aglycone (FIG. 8D), where R is a trimethylsilyl.

FIG. 9 shows an assessment of uptake of glycoconjugates via glucose transporters using fluorescent probe 2-NBDG as competitor in Caco-2 cells. Transporter-mediated uptake of the tested compounds (50 μM, 40 min) resulted in decreased intracellular fluorescence (as measured by flow cytometry) compared to that of cells treated with 2-NBDG (100 μM) alone. Co-treatment with glucose (10 mM) and glucose transporter (GLUT1) inhibitor phloretin (100 μM, 30 min) are employed as positive controls. Experiments were conducted in triplicate and values shown are averages±standard deviation. Statistical analysis: ** p<0.01, *** p<0.001 as compared to the control.

FIG. 10 shows relative amounts of cell-surface GLUT1 in human colon adenocarcinoma Caco-2 cells and normal CCD-18co colon fibroblasts. Average fluorescence values are shown in the graph to the left. Flow cytometry histograms following immunostaining of cell-surface GLUT1 with rabbit anti-GLUT1 and AlexaFluor488-conjugated antibodies are shown on the right. Experiments were conducted in triplicate and values plotted as the average of the median values from the flow cytometry histogram ±standard deviation. Values are statistically different (p<0.01).

### DESCRIPTION OF PREFERRED EMBODIMENTS

Prochelation strategies, in which the chelator is activated in response to a triggering event, increase the selectivity of biologically active chelators, and are therefore addressing a contemporary challenge in the design of chelation approaches that target conditions, such as cancer.

As used herein, the term “chelator” refers to a compound or a moiety that is capable of coordinating (or binding) a metal ion in a polydentate (e.g., coordination via two or more atoms of moieties) fashion. As such, the chelator may be referred to a polydentate ligand. The chelator may include donor atoms, such as S, N, or O, which are atoms in the chelator that bind to the metal. For example, a chelator of the present invention may be an iron chelator, which is a chelator that coordinates iron. In some embodiments, the iron chelator may be a tridentate ligand that has three donor atoms. However, it is to be understood that chelators typically have binding affinity for more than one metal ion; therefore, the iron chelator of the present invention can also bind other types of metal ions and does not necessarily bind iron exclusively.

As used herein, the term “prochelators” refers to a compound or a moiety that is transformed into a chelator following activation via a chemical reaction (e.g., via reduction or with or by another compound) or by an enzyme such as a reducing enzyme. The prochelators described herein may comprise a disulfide bond and at least two donor atoms. For instance, the prochelator comprises a disulfide bond linked to a chelator that contains donor atoms. An “iron prochelator” is a prochelator that, when activated, is transformed into an iron chelator.

When describing a chemical reaction, the terms “initiating”, “contacting” and “reacting” are used interchangeably herein, and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

As known to one of ordinary skill in the art, reducing conditions, or reducing environments, refer to conditions in which oxidative species are removed or kept at low levels to prevent oxidation. Reducing species, such as thiols or reducing peptides and enzymes, may be utilized to maintain the reducing conditions. As known to one of ordinary skill in the art, the intracellular space of a cell is a reducing environment, namely, due to the presence of reducing species and the slightly acidic conditions where the intracellular pH is about 6.8.

“Alkyl” refers to a saturated linear monovalent hydrocarbon moiety of one to twelve, typically one to six, carbon atoms or a saturated branched monovalent hydrocarbon moiety of three to twelve, typically three to six, carbon atoms. Examples of alkyl groups include, but are not limited to, methyl, ethyl, n-propyl, 2-propyl, tert-butyl, pentyl, and the like.

“Aryl” refers to a monovalent mono-, bi- or tricyclic aromatic hydrocarbon moiety of 6 to 15 ring atoms. The aryl may be optionally substituted with one or more substituents within the ring structure, referred to herein as a substituted aryl. When two or more substituents are present in an aryl group, each substituent is independently selected.

The term “heteroaryl” means a monovalent monocyclic or bicyclic aromatic moiety of 5 to 12 ring atoms containing one, two, or three ring heteroatoms selected from N, O, or S, the remaining ring atoms being C. The heteroaryl ring is optionally substituted independently with one or more substituents. Exemplary heteroaryls include, but are not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

The term “ion stabilizing group” refers to a moiety whose presence in the molecule increases the stability of the ion relative to the absence of such a group. One skilled in the art can readily determine whether a substituent or a moiety is an ion stabilizing group.

“Protecting group” refers to a moiety, except alkyl groups, that when attached to a reactive group in a molecule masks, reduces or prevents that reactivity. Examples of protecting groups can be found in T. W. Greene and P. G. M. Wuts, Protective Groups in Organic Synthesis, 3^{rd} edition, John Wiley & Sons, New York, 1999, and Harrison and Harrison et al., Compendium of Synthetic Organic Methods, Vols. 1-8 (John Wiley and Sons, 1971-1996), which are incorporated herein by reference in their entirety. Representative hydroxy protecting groups include acyl groups, benzyl and trityl ethers, tetrahydropyranyl ethers, trialkylsilyl ethers and allyl ethers. Representative amino protecting groups include, formyl, acetyl, trifluoroacetyl, benzyl, benzyloxycarbonyl (CBZ), tert-butoxycarbonyl (Boc), trimethyl silyl (TMS), 2-trimethylsilyl-ethanesulfonyl (SES), trityl and substituted trityl groups, allyloxycarbonyl, 9-fluorenylmethyloxycarbonyl (FMOC), nitro-veratryloxycarbonyl (NVOC), and the like.

Redox-Activated Iron Prochelators

As previously discussed, Inventors have reported novel redox-activated prochelators as described below. In some embodiments, the chelating compound comprises a molecule according to Formula I or Formula IA below:

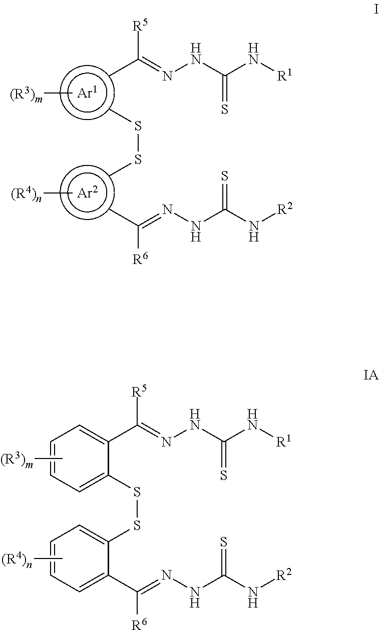

In some embodiments, m is an integer from 0 to 4. In some embodiments, n is an integer from 0 to 4. In some embodiments, Ar¹ is aryl or heteroaryl. In some embodiments, Ar² is aryl or heteroaryl. In some embodiments, R¹ comprises hydrogen, an alkyl, an aryl, an electron withdrawing group, or an ion stabilizing group. In some embodiments, R² comprises hydrogen, an alkyl, an aryl, an electron withdrawing group, or an ion stabilizing group. In some embodiments, R⁵ comprises a hydrogen, an alkyl, an aryl, an electron withdrawing group, or an ion stabilizing group. In some embodiments, R⁶ comprises a hydrogen, an alkyl, an aryl, an electron withdrawing group, or an ion stabilizing group. In some embodiments, R³ comprises an alkyl, a halide, an electron withdrawing group, or an ion stabilizing group. In some embodiments, R⁴ comprises an alkyl, a halide, an electron withdrawing group, or an ion stabilizing group. In some embodiments, R¹ is hydrogen, methyl or phenyl. In some embodiments, R² is hydrogen, methyl or phenyl.

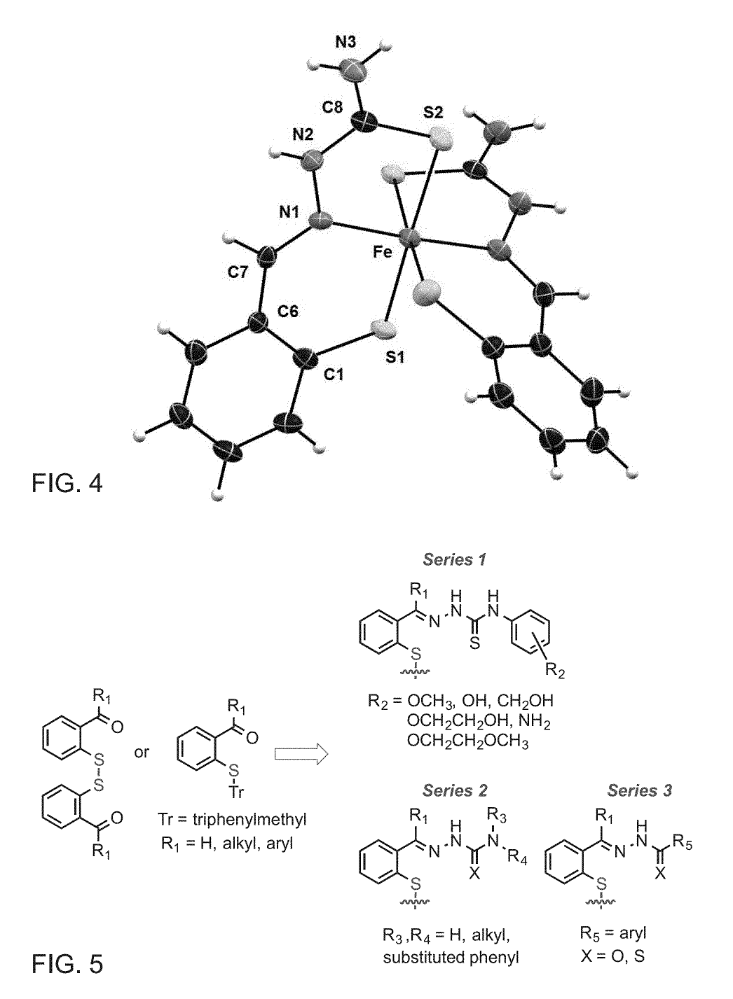

As shown in FIG. 3, the redox-activated prochelators are activated in reducing conditions. Chelators (e.g., TC1SH, TC2SH, TC3SH) are released upon reduction of the disulfide bond. The reduced species (e.g., TC1SH, TC2SH, TC3SH) are tridentate iron chelators forming complexes of 2:1 ligand-to-metal stoichiometry. Magnetic measurements and X-ray crystallographic data on the resulting iron complexes (see FIG. 4, BF₄⁻ counterion not shown) indicate that these chelators stabilize iron centers as low-spin Fe(III) species. EPR experiments in whole leukemia cells provided direct evidence for both the reduction/activation of (TC1S)₂ as well as the intracellular formation of the low-spin Fe(III) complex [(TC1S)₂Fe]⁺ as documented by its distinct spectroscopic features. The present invention is not limited to chelators that stabilize iron centers as low-spin Fe(III) species.

In some embodiments, prochelators of the present invention are reduced at half-cell potentials between −160 and −220 mV (vs SHE at 25° C.). In some embodiments, compounds of the invention are reduced at half-cell potentials of about −150 mV or lower, e.g., −160 mV or lower, e.g., −180 mV or lower, e.g., about −200 mV or lower. The present invention is not limited to the aforementioned reducing conditions.

Note that in some embodiments, the prochelator comprises a thiosemicarbazone. However, the present invention is not limited to thiosemicarbazones as any appropriate chelating agent may be use in lieu of the thiosemicarbazone. Other examples include, but are not limited to, semicarbazone, aroylhydrazone, desferrioxamine (DFO), deferiprone (DFP), hydroxypyridinone, deferasirox (DFX), desferrithiocin (DFT), or analogs thereof. In some embodiments, the thiosemicarbazone scaffold may be further modified, for examples, as described in Example 1, and FIGS. 5-7.

Glycoconjugated Prochelators

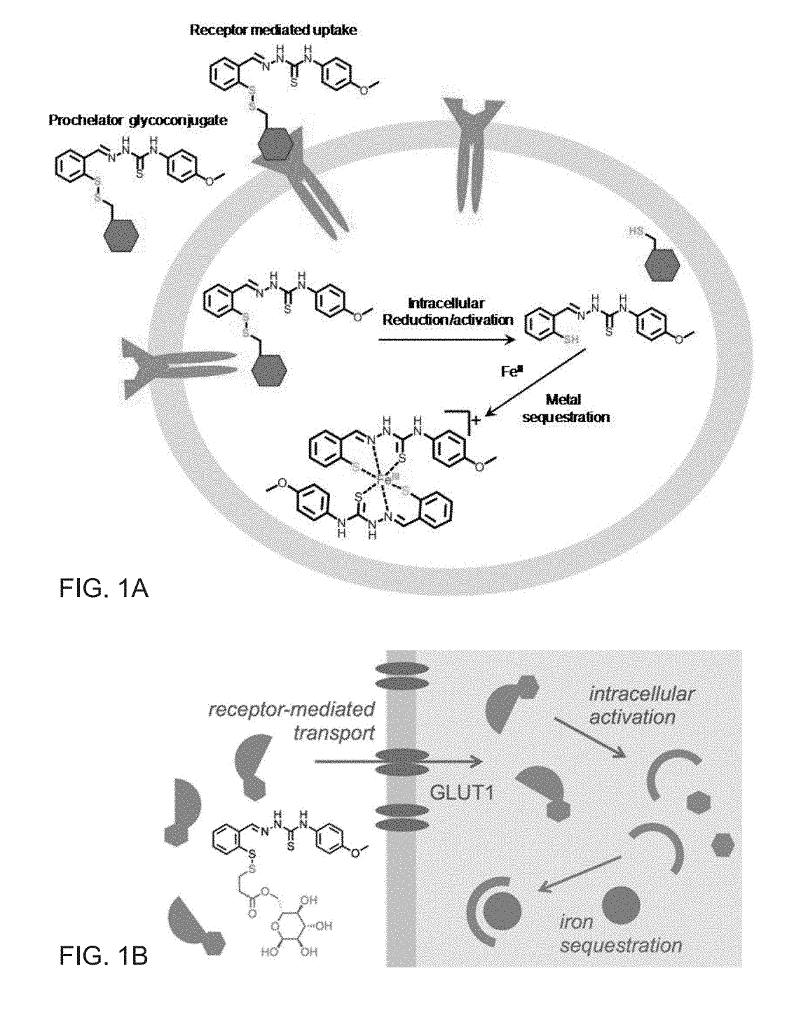

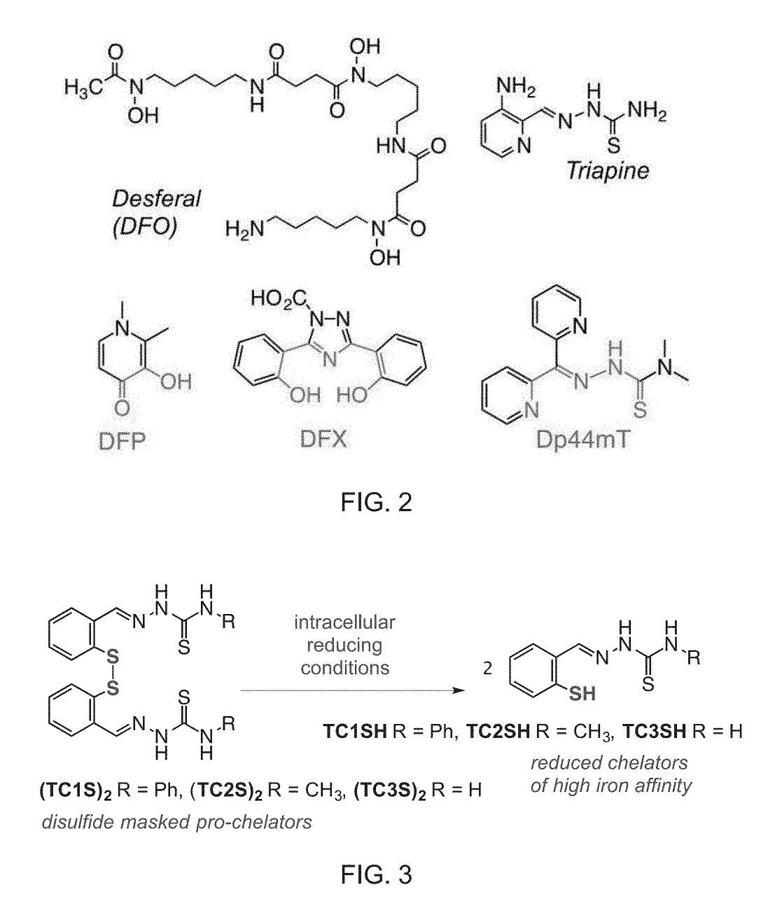

Referring now to FIGS. 1A-11, the present invention features glycoconjugated prochelators and methods of synthesizing said prochelators. As previously discussed, the glycoconjugated prochelators are taken up by cells via the glucose transporter and activated in reducing conditions so as to sequester iron. The glycoconjugated prochelators of the present invention may be used to target malignant cells (preferentially over normal cells). As such, the present invention also features methods of reducing or eliminating proliferation of a cell and methods of treating conditions associated with iron dysregulation (e.g., methods of treating a cancer) by treating the cells or patients with glycoconjugated prochelators of the present invention.

In some embodiments, the present invention features a compound comprising a prochelator conjugated to a carbohydrate moiety. The prochelator may be an iron prochelator. In one embodiment, the iron prochelator may comprise a disulfide bond and at least two donor atoms. The donor atoms may independently be an S, N, or O atom. In some embodiment, the iron prochelator may be linked to the carbohydrate moiety by the disulfide bond. Additional inker units may be included to link the disulfide bond to the carbohydrate moiety. Non-limiting examples of the linker unit between the disulfide bond and the carbohydrate moiety include an aliphatic chain, a polyether chain, a peptide chain, linkers containing more rigid aromatic moieties, or derivatives thereof. In some embodiments, the iron prochelator may comprise any one of the following:

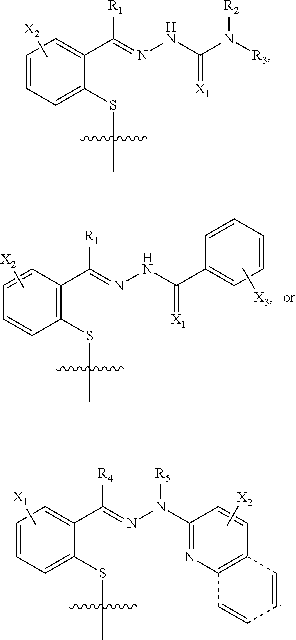

In some embodiments, R₁ may be a Ph, pyridyl, p-CF₃-Ph, p-NO₂-Ph, CCl₃ or CF₃. In some embodiments, R₂, R₃, R₄ may be independently an H, alkyl, aryl, or substituted aryl. In other embodiments, R₅ may be an H or alkyl. In other embodiments, X₁ is an O or S. In some other embodiments, X₂ and X₃ may be independently an H, alkyl, alkoxy, halide, CF₃, or NO₂.

In some embodiments, the carbohydrate moiety may be linked to the prochelator at a C2 carbon, C6 carbon, or at a position other than the C2 or C6 position of the carbohydrate moiety. For example, the carbohydrate moiety may be linked to the prochelator at its C2 carbon via an amidic linkage. In another example, the carbohydrate moiety may be linked to the prochelator at its C6 carbon via an ester linkage.

Without wishing to limit the present invention to a particular theory or mechanism, the carbohydrate moiety enables uptake of the compound into a cell via a glucose transporter protein. The prochelator can be activated upon entry into an intracellular space of the cell. In one embodiment, the prochelator may be activated by reduction to become a chelator. Again, without wishing to limit the present invention, the chelator can inhibit proliferation of a cell via sequestration of iron by the chelator. For example, the iron prochelators described herein can be activated by reduction of the disulfide bond to become an iron chelator. The iron chelator is a tridentate ligand that can inhibit proliferation of the cell via coordination of the donor atoms to iron, thereby sequestering iron. In some preferred embodiments, the cell may be a cancer cell, such as colorectal, breast, or pancreatic cancer cells.

According to other embodiments, the present invention features a method of synthesizing a glycoconjugated compound. The method may comprise initiating amidic or ester coupling of pyridyl disulfide crosslinker with a carbohydrate moiety to produce a cross-linked carbohydrate, and initiating disulfide exchange of the cross-linked carbohydrate with a chelator to link the chelator to the carbohydrate moiety via a disulfide bond, thereby producing the glycoconjugated compound. In one embodiment, the pyridyl disulfide crosslinker may be a according to the following:

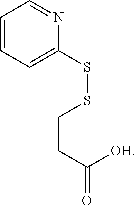 [SMILES: O=C(O)CCSSc1ccccn1]

However, the crosslinker is not limited to pyridyl disulfide, and any other disulfide crosslinker may be used. In some embodiments, the disulfide bond may further include a linker unit to the carbohydrate moiety. For instance, the linker unit between the disulfide bond and the carbohydrate moiety may be an aliphatic chain, a polyether chain, a peptide chain, linkers containing more rigid aromatic moieties, or derivatives thereof. In the example of the pyridyl sulfide above, the linker unit comprises an ester linkage.

Examples of chelators that may be used in the compounds and methods described herein include, but are not limited to, thiosemicarbazone, semicarbazone, aroylhydrazone, desferrioxamine (DFO), deferiprone (DFP), hydroxypyridinone, deferasirox (DFX), desferrithiocin (DFT), or analogs thereof. In other embodiments, the chelator may be according to any one of the following:

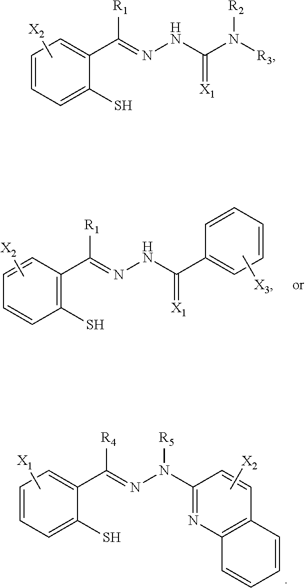

In one embodiment, R₁ may be a Ph, pyridyl, p-CF₃-Ph, p-NO₂-Ph, CCl₃ or CF₃. In another embodiment, R₂, R₃, R₄ may be independently an H, alkyl, aryl, or substituted aryl. In some embodiments, R₅ may be an H or alkyl. In other embodiments, X₁ is an O or S. In some other embodiments, X₂ and X₃ may be independently an H, alkyl, alkoxy, halide, CF₃, or NO₂. For example, the chelator may be a thiosemicarbazone according to the formula:

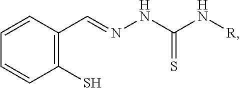

where R=Ph or alkoxy phenyl.

In other embodiments, the carbohydrate moiety may comprise a glucose molecule, a mannose molecule, or a sugar derivative. As used herein, the term “sugar derivative” refers to compounds having a saccharide structure, which is known to one of ordinary skill in the art, or to compounds having a structure similar to saccharides. Non-limiting examples of sugar derivatives include 2-deoxyglucose (2-DG) and amino sugars such as glucosamine.

Methods of Use

“A therapeutically effective amount” means the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Treating” or “treatment” of a disease includes: (1) preventing the disease, i.e., causing the clinical symptoms of the disease not to develop in a mammal that may be exposed to or predisposed to the disease but does not yet experience or display symptoms of the disease; (2) inhibiting the disease, i.e., arresting or reducing the development of the disease or its clinical symptoms; or (3) relieving the disease, i.e., causing regression of the disease or its clinical symptoms.

According to some embodiments, the glycoconjugated compounds described herein may be use in methods of inhibiting proliferation of a cancer cell. In one embodiment, the method may comprise introducing to the cancer cell any one of the glycoconjugated compounds. Without wishing to limit the invention to a particular theory or mechanism, the glyconjugated prochelator compound may be introduced into the cell by being uptaken by a glucose transporter protein. These glucose transporter proteins are overexpressed in cancer cells. Upon reduction of the disulfide bond, the glycoconjugated compound can release high-affinity chelators, which can sequester iron, thereby depriving the cell of iron and inhibiting its proliferation.

The present invention also features methods of treating a clinical condition associated with an iron dysregulation in a subject in need of such treatment. In one embodiment, the method may comprise administering to the subject a therapeutically effective amount of any of the compounds comprising a prochelator conjugated to a carbohydrate moiety as described herein. In some embodiments, the clinical condition is a cancer, however the present invention is not limited to cancer and the clinical condition may be any condition that is associated with iron dysregulation in a cell. Without wishing to limit the invention to a particular theory or mechanism, the glyconjugated prochelator compound, when administered to the subject, can release high-affinity chelators upon reduction of the disulfide bond. The high-affinity chelators can sequester iron, thereby treating the iron dysregulation condition.

The present invention shows that disulfide-based glycoconjugate prochelation strategies offer viable options for targeting of intracellular metal ions upon preferential uptake by cells presenting overexpression of glucose transporters. In addition to colorectal cancer, potential applications of this approach are relevant to several other human cancer phenotypes, including breast, pancreatic and lung carcinomas.

According to other embodiments, the present invention also features methods of increasing water solubility of an iron prochelator (e.g., a thiosemicarbazone or other appropriate prochelator). In some embodiments, the method comprises conjugating a carbohydrate moiety or a derivative thereof to an iron chelator, thereby producing a glycoconjugated prochelator (e.g., a glycoconjugated prochelator according to the present invention). The carbohydrate moiety and iron chelator utilized in this method may be according to any of the carbohydrate moieties and iron chelators described herein. Without wishing to limit the invention to a particular theory or mechanism, the carbohydrate moiety can increase water solubility of the prochelator.

Administration and Pharmaceutical Composition

In some embodiments, the present invention features pharmaceutical compositions comprising at least one glycoconjugated prochelator compound of the invention, or an individual isomer, racemic or non-racemic mixture of isomers or a pharmaceutically acceptable salt or solvate thereof, together with at least one pharmaceutically acceptable carrier, and optionally other therapeutic and/or prophylactic ingredients.

“Pharmaceutically acceptable carrier” refers to a carrier that is useful in preparing a pharmaceutical composition that is generally safe, non-toxic and neither biologically nor otherwise undesirable, and that is acceptable for veterinary use as well as human pharmaceutical use.

“Pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

In some embodiments, the compounds of the invention are administered in a therapeutically effective amount by any of the accepted modes of administration for agents that serve similar utilities. One of ordinary skill in the art of treating such diseases is typically able, without undue experimentation and in reliance upon personal knowledge and the disclosure of this application, to ascertain a therapeutically effective amount of the compounds of the invention.

In some embodiments, compounds of the invention are administered as pharmaceutical formulations including those suitable for oral (including buccal and sub-lingual), rectal, nasal, topical, pulmonary, vaginal, or parenteral (including intramuscular, intraarterial, intrathecal, subcutaneous and intravenous) administration or in a form suitable for administration by inhalation or insufflation.

In some embodiments, a compound or compounds of the invention, together with one or more conventional adjuvants, carriers, or diluents, can be placed into the form of pharmaceutical compositions and unit dosages. In some embodiments, the pharmaceutical compositions and unit dosage forms can be comprised of conventional ingredients in conventional proportions, with or without additional active compounds or principles, and the unit dosage forms can contain any suitable effective amount of the active ingredient commensurate with the intended daily dosage range to be employed. In some embodiments, the pharmaceutical compositions can be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use.

In some embodiments, the compounds of the invention can be formulated in a wide variety of oral administration dosage forms. The pharmaceutical compositions and dosage forms may comprise a compound or compounds of the invention or pharmaceutically acceptable salts thereof as the active component. In some embodiments, the pharmaceutically acceptable carriers can be either solid or liquid. Solid form preparations include powders, tablets, pills, capsules, cachets, suppositories, and dispersible granules. A solid carrier may be one or more substances which can also act as diluents, flavoring agents, solubilizers, lubricants, suspending agents, binders, preservatives, tablet disintegrating agents, or an encapsulating material. Suitable carriers may include but are not limited to magnesium carbonate, magnesium stearate, talc, sugar, lactose, pectin, dextrin, starch, gelatine, tragacanth, methylcellulose, sodium carboxymethylcellulose, a low melting wax, cocoa butter, and the like. The term “preparation” is intended to include the formulation of the active compound with encapsulating material as carrier, providing a capsule in which the active component, with or without carriers, is surrounded by a carrier, which is in association with it. Similarly, cachets and lozenges are included. Tablets, powders, capsules, pills, cachets, and lozenges can be as solid forms suitable for oral administration.

Other forms suitable for oral administration include liquid form preparations including emulsions, syrups, elixirs, aqueous solutions, aqueous suspensions, or solid form preparations which are intended to be converted shortly before use to liquid form preparations. Emulsions can be prepared in solutions, for example, in aqueous propylene glycol solutions or may contain emulsifying agents, for example, such as lecithin, sorbitan monooleate, or acacia. Aqueous solutions can be prepared by dissolving the active component in water and adding suitable colorants, flavors, stabilizers, and thickening agents. Aqueous suspensions can be prepared by dispersing the finely divided active component in water with viscous material, such as natural or synthetic gums, resins, methylcellulose, sodium carboxymethylcellulose, and other well known suspending agents. Solid form preparations include solutions, suspensions, and emulsions, and can contain, in addition to the active component, colorants, flavors, stabilizers, buffers, artificial and natural sweeteners, dispersants, thickeners, solubilizing agents, and the like.

In some embodiments, the compounds of the invention can also be formulated for parenteral administration (e.g., by injection, for example bolus injection or continuous infusion) and can be presented in unit dose form in ampoules, pre-filled syringes, small volume infusion or in multi-dose containers with an added preservative. The compositions can take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and can contain formulatory agents such as preserving, wetting, emulsifying or suspending, stabilizing and/or dispersing agents. Alternatively, the active ingredient can be in powder form, obtained by aseptic isolation of sterile solid or by lyophilization from solution for constitution before use with a suitable vehicle, e.g., sterile, pyrogen-free water. In some embodiments, the compounds of the invention can be formulated for topical administration to the epidermis as ointments, creams or lotions, or as a transdermal patch. Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatine and glycerine or sucrose and acacia; and mouthwashes comprising the active ingredient in a suitable liquid carrier.

In some embodiments, the compounds of the invention can be formulated for administration as suppositories. For example, a low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify. In some embodiments, the compounds of the invention can also be formulated for vaginal administration. Pessaries, tampons, creams, gels, pastes, foams or sprays containing in addition to the active ingredient such carriers as are known in the art to be appropriate. In some embodiments, the compounds of the invention can be formulated for nasal administration. The solutions or suspensions are applied directly to the nasal cavity by conventional means, for example, with a dropper, pipette or spray. The formulations can be provided in a single or multidose form. In the latter case of a dropper or pipette, this can be achieved by the patient administering an appropriate, predetermined volume of the solution or suspension. In the case of a spray, this can be achieved for example by means of a metering atomizing spray pump.

Other suitable pharmaceutical carriers and their formulations are described in Remington: The Science and Practice of Pharmacy 1995, edited by E. W. Martin, Mack Publishing Company, 19th edition, Easton, Pa.

The term “prodrug” refers to a pharmacologically substantially inactive derivative of a parent drug molecule that requires biotransformation, either spontaneous or enzymatic, within the organism to release the active drug. Prodrugs are variations or derivatives of the compounds of this invention, which have groups cleavable under metabolic conditions. Prodrugs become the compounds of the invention which are pharmaceutically active in vivo when they undergo solvolysis or reduction or other reaction eliciting activation under physiological conditions or undergo enzymatic processing. Prodrug compounds of this invention may be called single, double, triple etc., depending on the number of biotransformation steps required to release the active drug within the organism, and indicating the number of functionalities present in a precursor-type form. Prodrug forms often offer advantages of solubility, tissue compatibility, or delayed release in the mammalian organism (see, Bundgard, Design of Prodrugs, pp. 7-9, 21-24, Elsevier, Amsterdam 1985 and Silverman, The Organic Chemistry of Drug Design and Drug Action, pp. 352-401, Academic Press, San Diego, Calif., 1992).

Prodrugs commonly known in the art include acid derivatives that are well known to one skilled in the art, such as, but not limited to, esters prepared by reaction of the parent acids with a suitable alcohol, or amides prepared by reaction of the parent acid compound with an amine, or basic groups reacted to form an acylated base derivative. Moreover, the prodrug derivatives of this invention may be combined with other features herein taught to enhance bioavailability. For example, a compound of the invention having free amino, amido, hydroxy or carboxylic groups can be converted into prodrugs. Prodrugs include compounds where an amino acid residue, or a polypeptide chain of two or more (e.g., two, three or four) amino acid residues, which are covalently joined through peptide bonds to free amino, hydroxy or carboxylic acid groups of compounds of the invention. The amino acid residues include the 20 naturally occurring amino acids and also include, 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone. Prodrugs also include compounds wherein carbonates, carbamates, amides and alkyl esters which are covalently bonded to the above substituents of a compound of the invention through the carbonyl carbon prodrug sidechain.

### EXAMPLES

The following are non-limiting examples of synthesizing the glycoconjugate prochelators of the present invention. It is to be understood that the examples are for illustrative purposes only and are not intended to limit the present invention in any way. Equivalents or substitutes are within the scope of the invention.

Materials and Instruments

2-(N-(7-Nitrobenz-2-oxa-1,3-diazol-4-yl)amino)-2-deoxyglucose (2-NBDG), 3-(2-pyridyldithio)propionic acid (compound 1), TC₄-SH, 3,4,5,6-tetrakis((trimethylsilyl)oxy) glucopyranose, and 3,4,5,6-tetrakis((trimethylsilyl)oxy)mannopyranose were prepared. Human holotransferrin (Aldrich) was obtained commercially and used as received. AlexaFluor®488 goat anti-rabbit IgG was purchased from Fisher and used and stored per manufacturer instructions. Rabbit polycolonal anti-GLUT1 antibody was purchased from VWR and used as specified.

Thin layer chromatography (TLC) was conducted on Silica Gel 60 F254 X plates. NMR spectra were recorded on Bruker AVIII 400 MHz and Bruker DRX-500 MHz NMR spectrometers. Chemical shifts are reported in parts per million (ppm, δ) with residual solvent peaks and/or TMS peak set as reference. Proton coupling patterns are described as singlet (s), doublet (d), triplet (t), quartet (q), multiplet (m), and broad (br). High-resolution mass spectra (HRMS) were recorded on Bruker 9.4 T Apex-Qh hybrid Fourier transfer ion-cyclotron resonance (FT-ICR) spectrometer in the Mass Spectrometry Facility at the University of Arizona Department of Chemistry and Biochemistry. UV-visible absorption spectra were obtained on an Agilent 8453 spectrophotometer. Absorption assays in 96-well plates were recorded on a BioTek Synergy™ 2 microplate reader.

Flow cytometric analysis was performed at the University of Arizona Cytometry Core Facility (Arizona Cancer Center/Arizona Research Laboratories) using a FACSCanto II flow cytometer (BDBiosciences, San Jose, Calif.) equipped with an air-cooled 15-mW argon ion laser tuned to 488 nm. The emission fluorescence of 2-NBDG and AlexaFluor®488 were detected and recorded through a 530/30 and bandpass filter in the FL1 channel. List mode data files consisting of 10,000 events gated on FSC (forward scatter) vs SSC (side scatter) were acquired and analysed using CellQuest PRO software (BD Biosciences, San Jose, Calif.). Appropriate electronic compensation was adjusted by acquiring cell populations stained with each dye/fluorophore individually, as well as an unstained control.

Synthesis of Glycoconjugated Prochelators

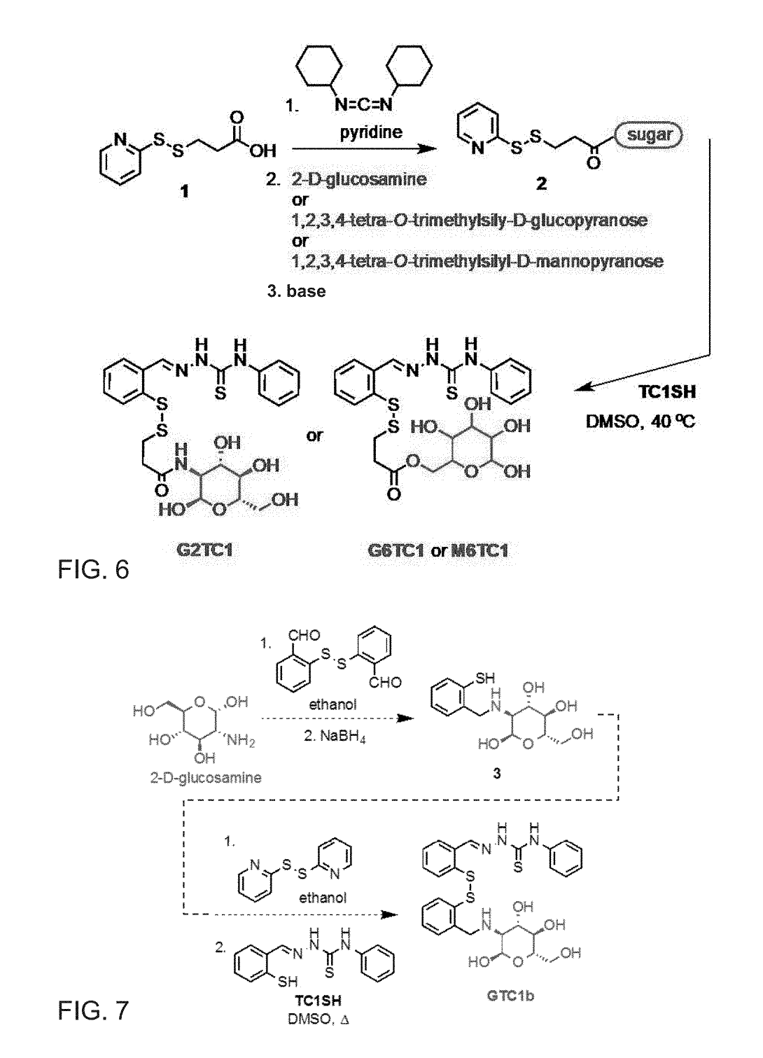

The disulfide bond of the prochelators was used as both the redox activation switch and the linking region for a carbohydrate (e.g., sugar) moiety. As shown in FIG. 6, glycoconjugates of TC1SH (e.g., G2TC1, G6TC1, M6TC1) were synthesized using the chemistry of 2-pyridyl disulfide crosslinkers. Amidic coupling of pyridyl disulfide 1 with D-glucosamine afforded key intermediate 2, a glycoconjugation partner.

Synthesis of 2: Compound 1 (1.00 g, 4.64 mmol) was dissolved in pyridine (4 mL) along with N,N′-dicyclohexylcarbodiimide (DCC, 1.15 g, 5.57 mmol) and stirred for 15 minutes. This solution was added slowly to a solution of D-glucosamine hydrochloride (3.01 g, 13.92 mmol) in aqueous NaOH (2.00 M, 2.30 mL). This solution was allowed to stir overnight then diluted with water (10 mL) to precipitate the urea product, which was removed by filtration. The resulting yellow solution was washed with diethyl ether, and then the volume of the aqueous fraction was reduced by rotary evaporation with minimal heating (below 40° C.). The product precipitated as a white solid which was collected on a fritted filter, washed with water and dried under vacuum (0.77 g, 58% yield). ¹H NMR (500 MHz, DMSO-d6) δ 8.46 (ddd, J=4.8, 1.9, 0.9 Hz, 1H), 7.87-7.82 (m, 1H), 7.79 (tt, J=7.0, 1.2 Hz, 2H), 7.24 (ddd, J=7.3, 4.8, 1.2 Hz, 1H), 6.42 (dd, J=4.6, 1.2 Hz, 1H), 4.95-4.86 (m, 2H), 4.61 (d, J=5.5 Hz, 1H), 4.41 (dd, J=6.4, 5.3 Hz, 1H), 3.59 (dddd, J=15.7, 7.2, 4.8, 2.4 Hz, 3H), 3.52-3.44 (m, 2H), 3.11 (ddd, J=9.8, 8.6, 5.3 Hz, 1H), 3.00 (t, J=7.3 Hz, 2H), 2.55 (td, J=7.2, 2.1 Hz, 2H). ¹³C NMR (126 MHz, DMSO) δ 170.39, 159.72, 150.00, 138.35, 121.58, 119.57, 91.01, 72.53, 71.57, 70.90, 61.57, 54.87, 35.11, 34.77. HRMS [M+Na]⁺ calculated: 399.07, found: 399.06542.

The substitution of the D-glucose moiety at the C2 position was selected (which may be tolerated well by the GLUT1 transporter). Disulfide exchange then afforded glycoconjugate G2TC1 in surprisingly good yield. Note that the present invention is not limited to substitution at the C2 position: other sugar-based molecules or derivatives may be used, including mannose, fructose, galactose and others, or the carbohydrate moieties may be conjugated at different positions, e.g., C1, C3, C4 and C6 positions, or others. For example, using a similar synthetic approach, a glucose moiety has also been introduced on the TC1 scaffold via an ester linkage to the C6 position of glucose and mannose.

Surprisingly, the glycoconjugate product was synthesized in good yield. Further, it was surprisingly discovered that the presence of the sugar-based conjugate did not necessarily interfere with the prochelator activation system (e.g., the —OH groups of glucose did not appear to interfere with the redox-directed activation of the prochelator). Interestingly, the introduction of the carbohydrate moieties led to a marked increase in the water solubility of the prochelators as compared to their unconjugated counterparts.

Without wishing to limit the present invention to any theory or mechanism, it is believed that the glycoconjugates of the present invention may be less toxic as compared to their unconjugated counterparts.

Again, without wishing to limit the present invention to any theory or mechanism, it is believed that the conjugates G2TC1 and G6TC1 present a mixed aliphatic/aromatic disulfide that may be reduced at more negative potentials when compared to the parent disulfide prochelator (TC1S)₂. In some embodiments, alternative conjugate designs may be employed in which the linkage remains an aromatic disulfide as in (TC1S)₂ (see FIG. 7). In some embodiments, a particular glycoconjugate prochelator may be synthesized so as to have a particular reduction property. For example, reductive amination of 2,2′-dithiodibenzaldehyde with D-glucosamine will lead to building block 3, which will be connected to the metal-binding unit via a 2-pyridyldisulfide intermediate. Compounds of this series (e.g., G2TC1b of FIG. 7) may therefore feature a more rigid linker and an aromatic disulfide switch. Similar routes involving the esterification of 2,2′-dithiodibenzoic acid with appropriately protected precursors (e.g., 1,2,3,4-tetra-O-trimethylsily-D-glucopyranose) can lead to C6-type conjugates.

### Example 1. Modifications of the thiosemicarbazone Scaffold

Example 1 describes modification (e.g., optimization) of thiosemicarbazone compounds, e.g., thiosemicarbazone disulfide compounds, e.g., compounds in the series of compounds TC1-4.

Many different modifications may be made while maintaining a general structure of thiosemicarbazone disulfide. In some embodiments, compounds are prepared through condensation of 2,2′-dithiodibenzaldehyde or diketone precursors with appropriate semicarbazides or hydrazides (see FIG. 5). In some embodiments, thiol precursors protected as thioethers are employed as alternative precursors. Upon deprotection of the thiol group, the condensation products will be then oxidized to disulfide prochelators using mild oxidants (e.g., air, I₂, aqueous H₂O₂/NaI). In some embodiments, the thiol chelators could be employed directly for the preparation of glycoconjugates.

For the thiosemicarbazones TC1 (R=phenyl) and TC4 (R=4-methoxyphenyl), in some embodiments, modifications may be employed to decrease its lipophilicity. In some embodiments, substituents may be introduced on the (TC1S)₂ to reduce its logP value (FIG. 5, Series 1). For example, in some embodiments, functionalization with polyethylene glycol (PEG) chains may be pursued.

Regarding Series 2 of FIG. 5, in some embodiments, the substitution of a thiocarbonyl with a carbonyl donor is employed. In some embodiments, substitutions may be important for toxicity. In some embodiments, all three substitutions on the ligand scaffold (R₁-R₃) may allow modulation of lipophilicity.

In some embodiments, condensation reactions with hydrazide precursors lead to the aroylhydrazones in Series 3 of FIG. 5. For example, substitutions on the aryl ring R₅ may be used. In some embodiments, aroylhydrazone precursors are used.

The present invention features constructs that incorporate a disulfide bond, which may be used as a prodrug trigger. For example, in some embodiments, all of the compounds in Series 1-3 of FIG. 5 may be employed as homodisulfide prochelators or as heterodisulfide glycoconjugates or also as conjugates with other targeting units.

In some embodiments, intracellular activation and iron chelation may be investigated using a calcein-based iron displacement assay. This assay may confirm the activation of the prochelators for iron scavenging, and may also indicate the timeframe for cellular entry and iron binding. For example, passive diffusion, activation and iron coordination occurred within minutes for (TC1S)₂ in suspended Jurkat cells. Data such as this may be collected for particular glycoconjugates to assess the duration of uptake and in particular to determine differences among systems carrying different connectivity (e.g., linker, position of glucose substitution) between the sugar and the prochelator units.

The present invention is not limited to the aforementioned synthesis pathways for glycoconjugate prochelators of the present invention. For example, the schemes of FIG. 6 and FIG. 7 are amenable to the modular assembly of prochelator conjugates of various binding units. Schemes that allow for additional or different conjugation positions may be employed. For example, synthetic routes of ester conjugation are available for the C1 position using a benzylated trichloroacetimidate as glycosyl donor, and for the C3 position by protecting the other positions using acetyl salicyloylchlorides. In some embodiments, longer linkers are used. For example, in some embodiments, PEGylated glucose precursors carrying a thiol group may be used.

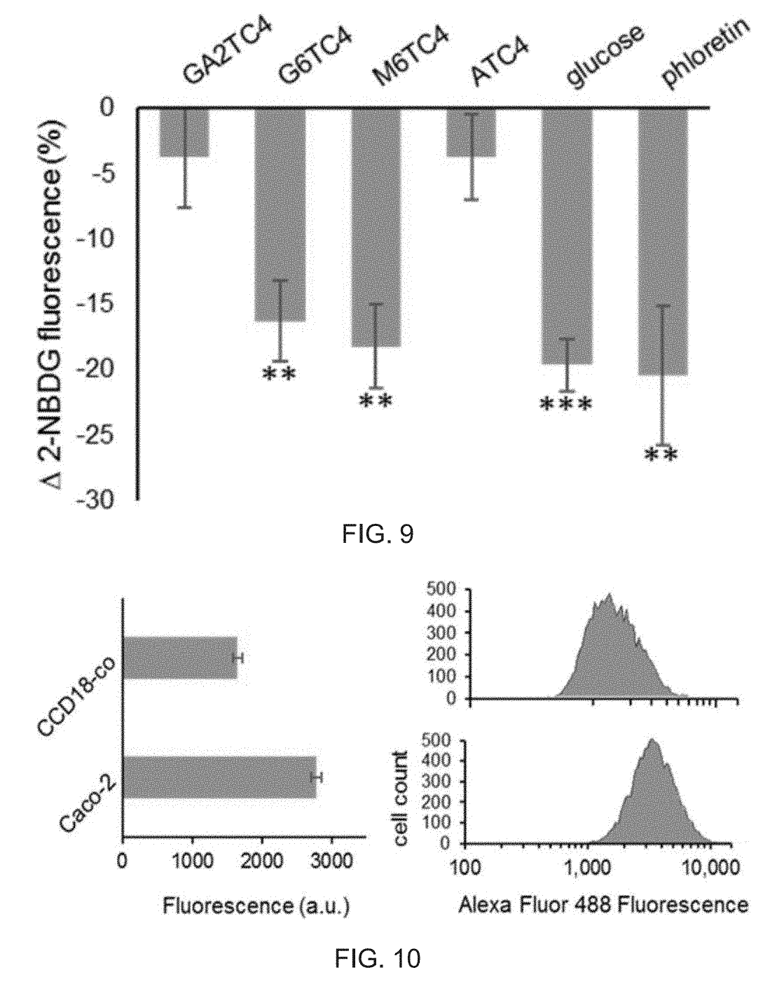

FIG. 9 shows competition assays investigating the mode of cellular entry of the glycoconjugates of the present invention, e.g., G2TC1, M6TC1, and G6TC1. The competition assays used fluorescently labeled glucose bioprobe 2-(N-(7-nitrobenz-2-oxa-1,3-diazol-4-yl)amino)-2-deoxyglucose (2-NBDG). After co-incubation of G2TC1 (25-100 μM, 30 min) and 2-NBDG (200 μM) in cultured MDA-MB-231 cells, measurements of intracellular fluorescence by flow cytometry showed a decreased uptake of the fluorescent glycoconjugate in the presence of G2TC1 in a concentration-dependent fashion. Interestingly, in a comparison of the three glycoconjugates, the ester-linked constructs at the C6 position (G6TC1 and M6TC1, 50 μM, 30 min) seemed to be better competitors of 2-NBDG (200 μM) when compared to G2TC1 (50 μM) in CACO-2 colorectal adenocarcinoma cells. The percent decrease in fluorescence in CACO-2 cells treated with 2-NBDG and G6TC1 is over three times the decrease due to G2TC1, an indication that selectivity and uptake may be dependent on the type and position of the linker. These experiments indicated that the glucose receptors are likely involved in cellular uptake of the glycoconjugate prochelators of the present invention. Without wishing to limit the present invention to any theory or mechanism, it is believed that the glycoconjugation may offer an advantageous increase in cancer cell specificity due to the conjugate being a substrate for the GLUT1 transporter protein and thus taking advantage of its overexpression in cancer cells as compared to normal cells.

### Example 2

Referring to FIG. 1A, a prochelation strategy required development of synthetic methods to connect carbohydrate moieties to thiosemicarbazone TC4-SH, an analog of chelator TC1-SH (featuring a phenyl group in place of a 4-methoxyphenyl group) and an antiproliferative thiosemicarbazones. The disulfide switch of reduction/activation in this family of compounds was selected for glycoconjugation through a heterodisulfide linkage. Substitutions at positions 2 and 6 of glucose or mannose moieties were then selected because they are tolerated well by glucose transporters. For instance, a 2-amino-2-deoxyglucose conjugate of adriamycin enters cells via a GLUT-mediated mechanism and several conjugates to the C6 position of glucose also undergo GLUT1-mediated uptake.

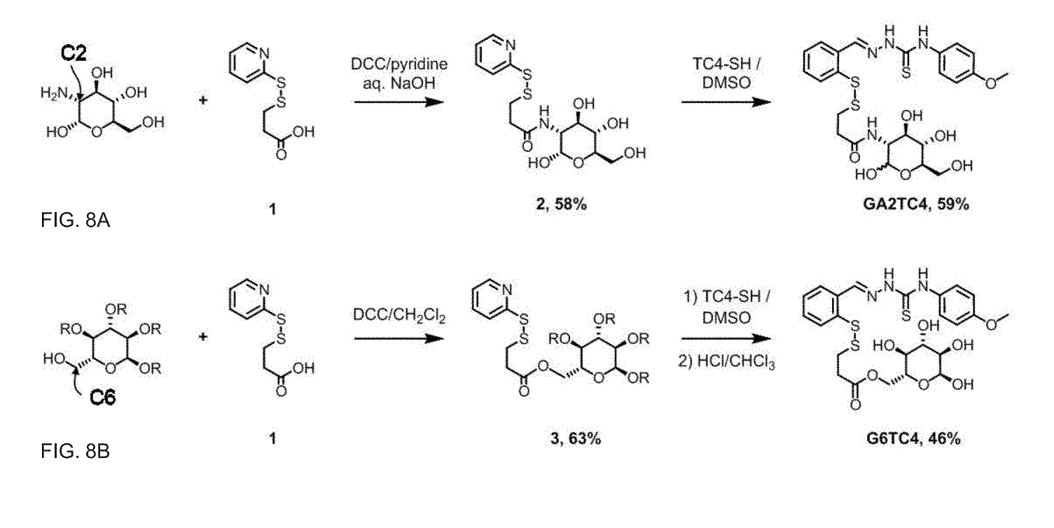

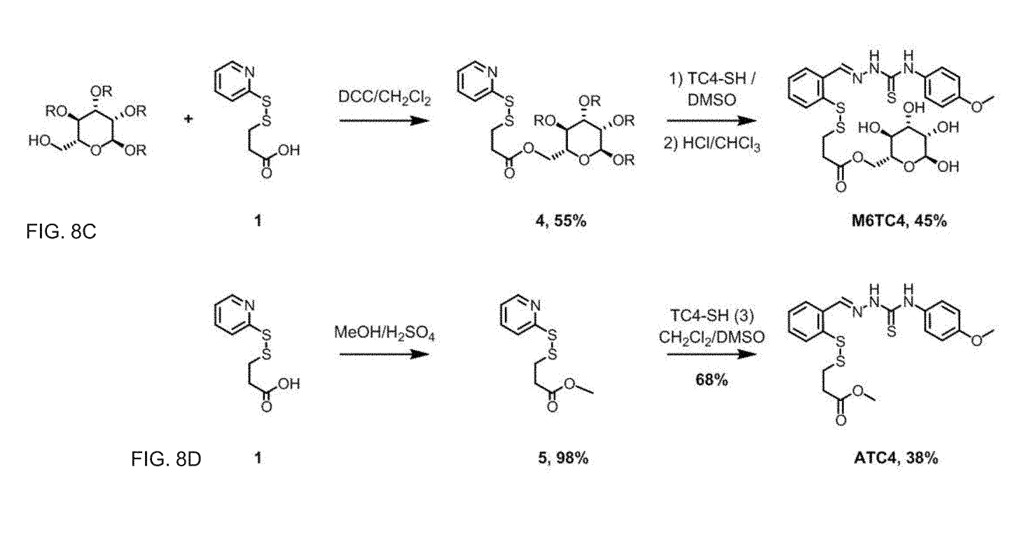

For the synthesis of the glucosamine conjugate GA2TC4 (FIG. 8A), the key compound 2 was prepared by amide linkage of 2-amino-2-deoxy-D-glucose (D-glucosamine) to crosslinker 1 using dicyclohexylcarboiimide (DCC) as a coupling reagent. Compound 2 is then used in excess in the presence of TC4-SH for the disulfide exchange reaction resulting in GA2TC4.

Synthesis of GA2TC4: A solution of 2 (300 mg, 1.08 mmol) in degassed DMSO (5.0 mL) was heated to 40° C. and a solution of TC4-SH (103.3 mg, 0.36 mmol) in degassed DMSO (2.00 mL) was added dropwise over 15 minutes. The solution was allowed to stir for 30 minutes and water (50.0 mL) was added to precipitate the product. The mixture was centrifuged to separate the precipitate, which was re-suspended in water (20 mL) and collected on a fritted filter, washed with water and dried under vacuum (118 mg, 59% yield). ¹H NMR (500 MHz, DMSO-d6) δ 11.94 (s, 1H), 9.95 (d, J=2.5 Hz, 1H), 8.66 (d, J=3.8 Hz, 1H), 8.34-8.18 (m, 1H), 7.91-7.72 (m, 2H), 7.63-7.17 (m, 5H), 6.94 (dd, J=7.7, 5.4 Hz, 2H), 6.58-6.37 (m, 1H), 5.02-4.77 (m, 2H), 4.60 (d, J=5.6 Hz, 1H), 4.41 (s, 1H), 3.77 (d, J=2.8 Hz, 3H), 3.64-3.55 (m, 2H), 3.53-3.40 (m, 2H), 3.16-3.04 (m, 1H), 2.95 (t, J=7.3 Hz, 2H), 2.51-2.49 (m, 2H). ¹³C NMR (126 MHz, DMSO) δ 176.23, 170.40, 140.82, 139.37, 136.62, 133.54, 130.89, 130.11, 128.85, 128.66, 128.18, 125.78, 125.68, 91.01, 72.54, 71.58, 70.91, 61.58, 54.87, 35.24, 34.36. HRMS [M+Na]⁺ calculated: 605.12; found: 605.1164. Extinction coefficient (5.0 mM TRIS buffer, 0.3% DMSO, pH 7.40) ϵ_{320 nm}=10.71 Lmol⁻¹ cm

Referring to FIGS. 9B and 9C, the synthesis of the conjugates featuring an ester linkage to the C6 alcohol on the hexose scaffold required trimethylsilyl (TMS) protection of the other hydroxyl groups of the sugar. Ester coupling with 1 in the presence of DCC and disulfide exchange with TC4-SH resulted in the TMS-protected glucose and mannose conjugates. Deprotection using HCl following chromatographic purification afforded the glucose ester-linked conjugate G6TC4 (FIG. 8B) and the mannose analog M6TC4 (FIG. 8C).

Synthesis of 3: 3,4,5,6-tetrakis((trimethylsilyl)oxy)glucopyranose (1.74 g, 3.70 mmol), 1 (875 mg, 4.07 mmoles), DCC (876 mg, 4.25 mmol) and dimethylaminopyridine (DMAP, 32.6 mg, 0.259 mmol) were combined in dry dichloromethane (12 mL) and allowed to stir under argon for 1.5 h. Dichloromethane (40 mL) was added to precipitate the urea side-product and the mixture was filtered through a Celite plug. The resulting solution was concentrated under vacuum and loaded onto a silica column. Flash chromatography using a hexanes/ethyl acetate gradient (5-25% ethylacetate) resulted in precursor 3 as a clear oil (1.60 g, 63% yield). ¹H NMR (499 MHz, CDC1₃) δ 8.47 (d, J=4.8, Hz, 1H), 7.74-7.56 (m, 2H), 7.09 (ddd, J=7.2, 4.8, 1.2 Hz, 1H), 5.00 (d, J=3.0 Hz, 1H), 4.38 (dd, J=11.8, 2.2 Hz, 1H), 4.05 (dd, J=11.8, 5.5 Hz, 1H), 3.90 (ddd, J=9.8, 5.5, 2.3 Hz, 1H), 3.78 (t, J=8.8 Hz, 1H), 3.48-3.29 (m, 2H), 3.04 (t, J=7.3 Hz, 2H), 2.88-2.70 (m, 2H), 0.15 (s, 9H), 0.15 (s, 9H), 0.14 (s, 9H), 0.13 (s, 9H). ¹³C NMR (126 MHz, CDCl₃) δ 171.39, 159.70, 149.66, 137.06, 120.74, 119.70, 93.87, 73.89, 73.75, 72.38, 69.85, 64.23, 33.75, 33.18, 1.24, 0.97, 0.45, 0.18, 0.15. HRMS [M+Na]⁺ calculated: 688.21; found: 688.2064.

Synthesis of G6TC4: Compound 3 (1.84 mg, 2.69 mmol) was dissolved in dry and degassed dichloromethane (15 mL). TC4-SH (284 mg, 0.890 mmol) was dissolved in degassed dimethyl sulfoxide (5.0 mL) and added dropwise to the flask containing 3. The reaction mixture was stirred for 1 h under argon, then the product was extracted using ethyl acetate (30 mL), and washed with water (3×15 mL) and brine (2×15 mL). The ethyl acetate layer was dried over sodium sulfate and evaporated under reduced pressure. The crude product was purified by flash chromatography using an ethyl acetate/hexanes gradient resulting in the TMS-protected product as a clear oil, which was susceptible to partial deprotection in NMR solvents. For full deprotection, the product was dissolved in chloroform (10 mL) and methanolic HCl (3 N, 25 μL) was added while stirring. The reaction progress was monitored by TLC and upon completion the solvent was removed by rotary evaporation. The resulting residue was dissolved in a minimal amount of methanol/acetone (5 mL), and the product was precipitated by addition of nanopure water (24 mL). The white precipitate was collected by centrifugation, washed with water (2×20 mL) and dried under vacuum resulting in an off-white sticky solid (254 mg, 49% yield). ¹H NMR (500 MHz, DMSO-d₆) δ 11.95 (s, 1H), 9.96 (s, 1H), 8.67 (s, 1H), 8.36-7.71 (m, 2H), 7.48 (t, J=7.7 Hz, 1H), 7.45-7.36 (m, 3H), 6.98-6.90 (m, 2H), 6.69-6.27 (m, 1H), 5.09 (dd, J=29.6, 5.6 Hz, 1H), 4.98-4.71 (m, 2H), 4.57-4.26 (m, 2H), 4.01 (ddd, J=15.8, 11.7, 6.5 Hz, 1H), 3.77 (s, 3H), 3.46-3.35 (m, 2H, partially hidden under H₂O peak), 3.16-3.10 (m, 1H), 3.08-3.00 (m, 1H), 2.99-2.87 (m, 2H), 2.82-2.71 (m, 2H). ¹³C NMR (126 MHz, acetone-d₆) δ 176.66, 171.07, 157.47, 140.26, 136.52, 133.33, 131.93, 130.33, 130.31, 129.79, 128.66, 128.63, 127.69, 126.05, 113.43, 94.49, 94.37, 74.24, 74.00, 71.80, 71.59, 71.32, 70.32, 67.92, 67.61, 64.42, 64.38, 59.68, 54.86, 33.59, 33.56, 32.89, 32.84, 29.45, 29.30, 29.24, 29.15, 29.09, 28.99, 28.84, 28.77, 28.68, 28.53, 19.97. HRMS [M+Na]⁺ calculated: 606.1; found: 606.1005. Extinction coefficient (5.0 mM TRIS buffer, 0.3% DMSO, pH 7.40) ϵ_{320 nm}=11.1 Lmol⁻¹ cm⁻¹.

Synthesis of 4: 3,4,5,6-tetrakis((trimethylsilyl)oxy)mannopyranose (1.25 g, 2.70 mmol), 3(2-pyridyldithio)propionic acid 1 (630 mg, 2.90 mmol), DCC (630 mg, 4.25 mmol) and DMAP (22.7 mg, 0.186 mmol) were combined, purged with argon, and dissolved in dry dichloromethane (10 mL). The contents were stirred for 1.5 h under argon, then the urea side-product was precipitated by addition of dichloromethane (30 mL) and filtered off. The resulting solution was concentrated under reduced pressure and loaded onto a silica column. Flash chromatography using a hexanes/ethyl acetate gradient resulted in precursor 4 as a clear oil (1.01 g, 55% yield). ¹H NMR (499 MHz, chloroform-d) δ 8.48 (ddd, J=4.9, 1.9, 1.0 Hz, 1H), 7.77-7.52 (m, 2H), 7.10 (ddd, J=7.3, 4.8, 1.2 Hz, 1H), 4.92 (d, J=2.1 Hz, 1H), 4.40 (dd, J=11.6, 2.0 Hz, 1H), 4.08 (dd, J=11.6, 5.8 Hz, 1H), 3.89-3.77 (m, 3H), 3.68-3.64 (m, 1H), 3.06 (t, J=7.2 Hz, 2H), 2.81 (td, J=7.2, 4.0 Hz, 2H), 0.17 (s, 9H), 0.15 (s, 9H), 0.14 (bs, 18H). ¹³C NMR (126 MHz, CDCl₃) δ 171.42, 149.69, 137.01, 120.69, 119.65, 95.53, 75.03, 71.97, 71.73, 68.44, 64.45, 33.84, 33.29, 0.74, 0.63, 0.33, −0.13. HRMS [M+Na]⁺ calculated: 688.21; found: 688.2063.

Synthesis of M6TC4: Compound 4 (630 mg, 0.922 mmol, 3 equiv) was dissolved in dry and degassed dichloromethane (6.0 mL). TC4-SH (98 mg, 0.31 mmoles mmol) was dissolved in degassed dimethyl sulfoxide (2.00 mL) and added dropwise to the flask containing 4. The reaction was stirred for 1 h under argon, then the product was extracted using ethyl acetate (15 mL), and washed with water (3×10 mL) and brine (2×10 mL). The ethyl acetate layer was dried with sodium sulfate and then evaporated under reduced pressure. The crude product was purified by flash chromatography using an ethyl acetate/hexanes gradient (0-25% ethyl acetate in hexanes) resulting in the TMS-protected product as a clear oil. Deprotection was carried out in chloroform (4 mL) and methanolic HCl (3 N, 25 μL). The reaction progress was monitored by TLC and upon completion the solvent was removed by rotary evaporation. The resulting residue was dissolved in a minimal amount of methanol/acetone (1-2 mL), and the product was precipitated by addition of nanopure water (13 mL). The white precipitate was collected by centrifugation, washed with water (2×10 mL) and dried under vacuum resulting in an off-white sticky solid (82 mg, 45% yield). ¹H NMR (500 MHz, DMSO-d₆) δ 11.95 (s, 1H), 9.97 (s, 1H), 8.67 (s, 1H), 8.27 (dd, J=7.8, 1.5 Hz, 1H), 7.81 (dd, J=8.0, 1.2 Hz, 1H), 7.53-7.46 (m, 1H), 7.45-7.37 (m, 3H), 6.94 (d, J=9.0 Hz, 2H), 6.43-6.19 (m, 1H), 4.98-4.83 (m, 2H), 4.68-4.51 (m, 2H), 4.33 (ddd, J=13.6, 11.6, 1.9 Hz, 1H), 4.11-3.94 (m, 1H), 3.77 (s, 3H), 3.71 (ddd, J=9.3, 7.1, 2.2 Hz, 1H), 3.57-3.50 (m, 2H), 3.41-3.35 (m, 1H), 3.30 (s, 1H), 3.01-2.92 (m, 2H), 2.74 (dd, J=7.2, 6.2 Hz, 2H). ¹³C NMR (126 MHz, acetone-d₆) δ 176.66, 157.47, 140.26, 136.52, 133.33, 131.93, 130.33, 130.31, 129.79, 128.66, 128.63, 127.69, 126.05, 113.43, 94.49, 94.37, 74.24, 74.00, 71.80, 71.59, 71.32, 70.32, 67.92, 67.61, 64.42, 64.38, 54.86, 33.59, 32.89. HRMS [M+Na]⁺ calculated: 606.1; found: 606.10061. Extinction coefficient (5.0 mM TRIS buffer, 0.3% DMSO, pH 7.40) ϵ_{320 nm}=19.8 Lmol⁻¹ cm⁻¹.

Referring to FIG. 8D, an aglycone that retains the general structure of the glycoconjugates but lacks the carbohydrate targeting moiety was prepared as a control compound. Specifically, the sugar motif was replaced with a methyl group, thus protecting the carboxylate and maintaining a scaffold that would be neutral at biological pH. Fisher esterification of 1 and a disulfide exchange reaction similar to those conducted for the other conjugates resulted in the desired compound ATC4.

Synthesis of 5: Compound 1 (200 mg, 0.930 mmol) was dissolved in freshly distilled methanol (5 mL) along with concentrated sulfuric acid (100 μL). The solution was heated to reflux and stirred under argon for 1 h. The solvent was removed by rotary evaporation. The residue was dissolved in ethyl acetate (5 mL), washed with a saturated bicarbonate solution (2×5 mL) and dried over anhydrous sodium sulfate. Precursor 5 was obtained after rotary evaporation of the solvent as a yellow residue (209 mg, 98% yield). ¹H NMR (499 MHz, chloroform-d, (matches that for reported compound from different procedure⁵) δ 8.49 (ddd, J=4.8, 1.8, 1.0 Hz, 1H), 7.75-7.62 (m, 2H), 7.12 (ddd, J=7.2, 4.8, 1.3 Hz, 1H), 3.71 (s, 3H), 3.07 (t, J=7.2 Hz, 2H), 2.79 (t, J=7.2 Hz, 2H). ¹³C NMR (126 MHz, DMSO) δ 171.90, 159.72, 149.69, 137.01, 120.79, 119.76, 77.38, 77.13, 76.87, 51.88, 33.63, 33.40.

Synthesis of ATC4: Compound 5 (113 mg, 0.493 mmol) was dissolved in dichloromethane (0.2 mL). TC4-SH (78.4 mg, 0.246 mmol) was dissolved in degassed dimethyl sulfoxide (2 mL) and added dropwise to the flask containing 5. The reaction mixture was stirred for 1 h under argon. The product was then extracted using ethyl acetate (10 mL), and washed with water (3×10 mL) and brine (2×10 mL). The ethyl acetate layer was dried over anhydrous sodium sulfate and the solvent was removed by rotary evaporation. The resulting solid was loaded onto a silica column, and flash chromatography using ethyl acetate/hexanes gradient (0-25% ethyl acetate in hexanes) afforded the desired product ATC4as an off-white powder (40 mg, 38%). ¹H NMR (499 MHz, DMSOI-d₆) ¹H NMR (499 MHz, Methanol-d₄) δ 11.96 (s, 1H), 9.97 (s, 1H), 8.68 (s, 1H), 8.28 (dd, J=7.8, 1.6 Hz, 1H), 7.81 (dd, J=8.0, 1.2 Hz, 1H), 7.57-7.31 (m, 4H), 7.00-6.85 (m, 2H), 3.78 (d, J=2.8 Hz, 3H), 3.60 (s, 3H), 2.98 (t, J=6.8 Hz, 2H), 2.76 (t, J=6.7 Hz, 2H). ¹³C NMR (126 MHz, DMSO-d6) δ 176.64, 171.92, 157.39, 140.46, 136.36, 134.11, 132.27, 130.97, 130.80, 128.59, 128.53, 127.56, 113.83, 55.75, 52.08, 40.54, 40.46, 40.37, 40.29, 40.20, 40.13, 40.04, 39.96, 39.87, 39.79, 39.70, 39.54, 33.57, 33.15. HRMS [M+Na]⁺ calculated: 458.06; found: 458.06355. Extinction coefficient (5.0 mM TRIS buffer, 0.3% DMSO, pH 7.40) ϵ_{340 nm}=10.1 Lmol⁻¹ cm⁻¹.

Solubility measurements. The effect of the sugar motif on the aqueous solubility of the synthesized prochelators was investigated by UV-Visible absorption spectroscopy. Determination of molar extinction coefficients of each compound in aqueous buffer (5.0 mM TRIS, pH 7.40) allowed measurement of their concentration at saturation. Stock solutions were prepared in DMSO and diluted in TRIS buffer (5.0 mM, pH 7.40). Buffer solutions containing identical amounts of DMSO (0.3% v/v) were used to obtain blank spectra. Molar extinction coefficients were obtained by recording UV-Vis spectra of a range of concentrations of the compounds (0.1-40 μM, depending on solubility) and then plotting the average of three absorbance values for each concentration at a specified wavelength. For solubility measurements, saturated solutions were centrifuged for 20 minutes at 3,200 rpm to remove any precipitate and concentrations were determined by UV-Vis spectroscopy. Absorbance values were found to be close to the linearity range of absorbances for each compound, therefore dilutions were not typically necessary. Measurements were done in a set of triplicates.

The sugar conjugates display moderate solubility in buffered aqueous solutions (Table 1), higher than the aglycone compound in all cases. In addition, estimated concentrations at saturation are 15-30 times larger than that for the symmetric disulfide system (TC4-S)₂ and thus highlight an advantageous aspect of the glycoconjugation approach for this family of prochelators.

TABLE 1
Aqueous solubility of disulfide prochelators

Concentration at saturation^{a}

| Compound | μM | mg/L |
|---|---|---|
| GA2TC4 | 53 ± 2 | 31 ± 1 |
| G6TC4 | 36 ± 5 | 21 ± 3 |
| M6TC4 | 25 ± 1 | 14 ± 1 |
| ATC4 | 11 ± 1 | 4.7 ± 0.5 |
| (TC4-S)₂ | <5 | <3 |

^{a}The concentration in a buffer solution (5.0 mM TRIS, pH 7.40) saturated with the indicated compound was measured by UV-Vis absorption spectroscopy after centrifugation and removal of any precipitate. All measurements were made in triplicate and values are plotted as the average of the triplicate set ± standard deviation between values.

The reported methods for the synthesis of 2-pyridyl disulfide derivatives of the selected carbohydrate units allowed preparation of glycoconjugates featuring an amidic linkage at the C2 position (GA2TC4) or an ester linkage at the C6 position (G6TC4 and M6TC4). The modular assembly of the prochelator components (from the linker to the carbohydrate to the metal-binding unit) can be amenable to the preparation of multiple series of compounds of this general design. The prepared glycoconjugates offer a considerable advantage as they are 15-30 times more soluble than the homodisulfide prochelator (TC4-S)₂ in neutral aqueous solutions.

Cell culture. Caco-2 (ATCC® HTB-37™) colorectal adenocarcinoma and CCD-18Co (ATCC® CRL-1459™) normal colon fibroblasts were cultured under a 5% CO₂ humidified atmosphere at 37° C. in Eagle's Minimal Essential Medium (EMEM) supplemented with 10% fetal bovine serum (FBS), glutamine (2 mM), sodium pyruvate (1 mM), sodium bicarbonate (1.5 g/L), penicillin (100 units/mL), streptomycin (100 μg/mL) and human holo-transferrin (1.25 μM (1 mg/10 mL) prior to use. Flow cytometric analysis was performed at the University of Arizona Cytometry Core Facility (Arizona Cancer Center/Arizona Research Laboratories) using a FACSCanto II flow cytometer (BD Biosciences, San Jose, Calif.) equipped with an air-cooled 15-mW argon ion laser tuned to 488 nm. List mode data files consisting of 10,000 events gated on FSC (forward scatter) vs SSC (side scatter) were acquired and analyzed using CellQuest PRO software (BD Biosciences, San Jose, Calif.). Appropriate electronic compensation was adjusted by acquiring cell populations stained with each dye/fluorophore individually, as well as an unstained control.

Competition assay of transporter-mediated uptake. Caco-2 cells were plated at 1×10⁶ cells per well in 6-well plates and allowed to adhere for 36 hours. Growth media were then removed and cells were incubated for 12 hours in glucose-free EMEM. Cells were then treated with EMEM containing 1 g/L glucose (for unstained control), glucose containing EMEM with 2-NBDG (100 μM) (for stained control) or a combination of 2-NBDG (100 μM) and test compounds (50 μM) for 40 minutes. As a positive control, after starvation cells were treated with 100 μM phloretin for 30 minutes, then treated with EMEM containing 100 μM 2-NBDG for 40 minutes. Cells were washed with PBS (1 mL) and detached by addition of Trypsin-EDTA (400 μL) and incubation for 3 minutes at 37° C. Following addition of EMEM (1 mL), the cell suspensions were centrifuged at 125 g for 10 minutes. The resulting pellets were suspended in PBS (0.5 mL) and transferred to a flow cytometry tube. Cells were stored on ice and analyzed by flow cytometry within 1 hour. Data are obtained as the average of three sets of geometric means of the flow cytometry histogram, and plotted as the percent difference from the control plus/minus the standard deviation between three values.

Cell-surface expression of GLUT1. Cells were plated at 1×10⁶ cells per well in 6-well plates and allowed to adhere overnight. Cells were detached by addition of 0.25% Trypsin-EDTA (400 μL) and incubation for 3 minutes at 37° C. Following addition of EMEM (1 mL), the cell suspensions were centrifuged at 125 g for 14 minutes. The resulting pellets were suspended in PBS (2 mL) containing bovine serum albumin (BSA, 1% w/v, to block non-specific binding of the antibody) then centrifuged at 125 g for another 14 minutes. The resulting pellet was suspended in 1% BSA (500 μL) and cells were counted. Cells were then treated with the primary antibody (at concentration determined from an optimized titration) and incubated on ice for 45 minutes. The cells were centrifuged for ten minutes at 125 g and washed with 1% BSA in PBS (1 mL) in order to remove excess antibody. The pellets were then re-suspended in 1% BSA in PBS (500 μL) and the secondary antibody was added and incubated for 30 minutes on ice in the dark. Cells were centrifuged after addition of 1% BSA in PBS (300 μL) for 10 minutes at 125 g. The resulting pellet was then suspended in 300 μL of 1% BSA in PBS (300 μL), stored on ice and analyzed by flow cytometry within 1 hour.

Cytotoxicity assays. The cytotoxicity of the prochelators were assessed using MTT (3-(4,5-dimethylthiazol-2-yl)-2,5-diphenyl-tetrazolium bromide) assays after 72-hour incubations (Table 2). In all cases, the prochelator conjugates are more toxic (lower IC₅₀ values) in the malignant Caco-2 cells than in the normal CCD18-co cells. Without wishing to limit the present invention to a particular theory or mechanism, the data indicate that disulfide-based prochelators are effective antiproliferative agents in this colorectal cancer model.

TABLE 2
Antiproliferative activity of TC4 prochelators
in Caco-2 and CCD18-co cell lines.

| | IC₅₀ (μM, 72 h)^{a} | | |
|---|---|---|---|
| | Caco-2 | CCD18-co | Therapeutic Index^{b} |
| GA2TC4 | 2.6 ± 0.3 | 22.5 ± 0.8 | 9 ± 1 |
| G6TC4 | 6.9 ± 0.4 | 76 ± 2 | 11 ± 1 |
| M6TC4 | 10.1 ± 0.3 | 62.2 ± 0.1 | 6.2 ± 0.4 |
| ATC4 | 8.1 ± 0.2 | 27.5 ± 0.4 | 3.4 ± 0.1 |

^{a}IC₅₀ values are obtained from MTT assays after 72-h exposure to the tested compounds. Values are the average of a triplicate set plus/minus standard deviation;
^{b}The therapeutic index is calculated as the ratio of IC₅₀ values in the normal cell line relative to the malignant cell line.

MTT (3-(4,5-dimethylthiazol-2-yl)-2,5-diphenyltetrazolium bromide) viability assays were conducted by standard methods with slight modifications. Cells were seeded at 4000 cells per well for Caco-2 and 10,000 cells per well for CCD18-co and allowed to attach for 24 h. Test compounds dissolved in DMSO were diluted in EMEM to the specified concentration (with final DMSO concentration limited to 0.1%). Cells were incubated in the presence of the test compounds for 72 h, then the MTT solution (4 mg/mL, 10 μL) was added to each well and incubated for 4 h. Following media removal, DMSO (100 μL) was added to each well to dissolve the purple formazan crystals and the plates were incubated for an additional 30 minutes. Absorption at 560 nm was recorded and data were analyzed using logarithmic fits to obtain IC₅₀ values. Each experiment was conducted in triplicate, and values are given as averages plus/minus standard deviation.

The aglycone prochelator ATC4 presents the lowest therapeutic index in this data set. This observation is consistent with an increased glucose uptake in cancer cells, and also with the relative expression levels of glucose transporter GLUT1 determined experimentally for the cell cultures under investigation (FIG. 10). Of the three glycoconjugate prototypes, the mannose construct M6TC4 is both the least toxic and least selective. Notably, as shown in FIG. 9, M6TC4 competed successfully with 2-NBDG for transporter-mediated uptake at the 50 μM concentration level, therefore the observed toxicity parameters could reflect lower affinity and/or overall uptake for the mannose unit relative to the glucose unit in this type of conjugates. In contrast, the glucosamine conjugate GA2TC4, which did not compete for uptake with 2-NBDG in the assay conditions, showed better toxicity and selectivity relative to the aglycone. Notably, the ester-linked glucose conjugate G6TC4, which competes strongly with 2-NBDG for uptake, maintains a toxicity level similar to the aglycone in Caco-2 cells but is significantly less toxic to normal CCD18-co cells. Overall, both glucose conjugates GA2TC4 and G6TC4 displayed an improved therapeutic index (three-fold or higher) in this comparative study of colorectal cell lines.

Overexpression of glucose transporters (e.g., GLUT1) is a negative prognostic biomarker in colorectal cancer patients therefore glycoconjugation could provide targeted access to malignant cells. In the present cell culture conditions, colon carcinoma Caco-2 cells were found to express GLUT1 at almost twice the level of normal colon fibroblasts CCD18-co. Consistently, the glycoconjugates are 6-11 times more toxic in Caco-2 cells than in CCD18-co cells. In contrast, the aglycone ATC4 is only 3 times more toxic in the cancer cell line. Notably, ester-linked C6-glucosyl prochelator G6TC4 competes aggressively with fluorescent glycoconjugate 2-NBDG for transporter-mediated cellular uptake and displays the highest therapeutic index among the tested glycoconjugates within this comparison in colorectal cell lines. Glycoconjugation did not impact dramatically the cytotoxicity of the constructs. Nevertheless, the glycoconjugate prochelators present improved therapeutic indexes in the tested colorectal cell lines.

As used herein, the term “about” refers to plus or minus 10% of the referenced number.

The disclosures of the following U.S. Patents are incorporated in their entirety by reference herein: U.S. Pat. No. 9,486,423, U.S. patent application Ser. No. 15/345,393, and U.S. Provisional Pat. Application No. 61/899,262.

For reference, the following documents are incorporated herein in their entirety: Akam, E. A., Tomat, E. “Targeting iron in colon cancer via glycoconjugation of thiosemicarbazone prochelators”. Bioconjugate Chem. 2016, 27, 1807-1812; Akam et al, 2014, Metallomics 6:1905-1912; Chang and Tomat, 2013, Dalton Trans. 42:7846-7849.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although it has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and for ease of review by the patent office only, and are not limiting in any way. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

### REFERENCES

Akam, E. A.; Chang, T. M.; Astashkin, A. V.; Tomat, E. “Intracellular reduction/activation of a disulfide switch in thiosemicarbazone iron chelators” Metallomics 2014, 6, 1905-1912.

Chang, T. M.; Tomat, E. “Disulfide/thiol switches in thiosemicarbazone ligands for redox-directed iron chelation” Dalton Trans. 2013, 42, 7846-7849.

Jung, M. E.; Dong, T. A.; Cai, X. L., Improved synthesis of 4-amino-7-nitrobenz-2,1,3-oxadiazoles using NBD fluoride (NBD-F). Tetrahedron Lett. 2011, 52, 2533-2535.

Widdison, W. C.; Wilhelm, S. D.; Cavanagh, E. E.; Whiteman, K. R.; Leece, B. A.; Kovtun, Y.; Goldmacher, V. S.; Xie, H. S.; Steeves, R. M.; Lutz, R. J.; Zhao, R.; Wang, L. T.; Blattler, W. A.; Chari, R. V. J., Semisynthetic maytansine analogues for the targeted treatment of cancer. Journal of medicinal chemistry. 2006, 49, 4392-4408.

Cui, Y. L.; Cheng, Z. D.; Mao, J. W.; Yu, Y. P., Regioselective 6-detrimethylsilylation of per-O-TMS-protected carbohydrates in the presence of ammonium acetate. Tetrahedron Lett. 2013, 54, 3831-3833.

Loccufier, J.; Schacht, E., Convenient Method for the Preparation of 3-(2-Pyridyl Dithio) Propionic-Acid N-Hydroxy Succinimide Ester. B Soc Chim Belg. 1988, 97, 535-539.

Akam, E. A., Tomat, E. “Targeting iron in colon cancer via glycoconjugation of thiosemicarbazone prochelators”. Bioconjugate Chem. 2016, 27, 1807-1812.

What is claimed is:

1. A compound comprising a prochelator conjugated to a carbohydrate moiety.

2. The compound of claim 1, wherein the prochelator is an iron prochelator.

3. The compound of claim 2, wherein the iron prochelator comprises a disulfide bond and at least two donor atoms.

4. The compound of claim 3, wherein the iron prochelator comprises any one of the following: 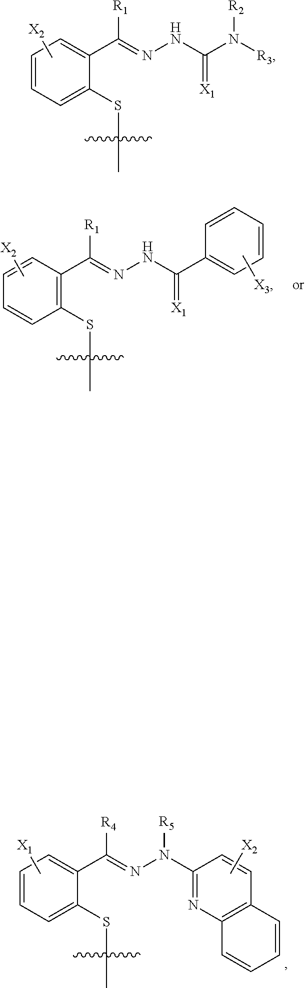
wherein R₁ is a Ph, pyridyl, p-CF₃-Ph, p-NO₂-Ph, CCl₃ or CF₃;
wherein R₂, R₃, R₄ are independently an H, alkyl, aryl, or substituted aryl;
wherein R₅ is an H or alkyl;
wherein X₁ is an O or S; and
wherein X₂ and X₃ are independently an H, alkyl, alkoxy, halide, CF₃, or NO₂.

5. The compound of claim 3, wherein iron prochelator is linked to the carbohydrate moiety by the disulfide bond.

6. The compound of claim 5, wherein the iron prochelator is activated upon entry into an intracellular space of the cell.

7. The compound of claim 6, wherein the iron prochelator is activated by reduction of the disulfide bond to become an iron chelator, wherein the iron chelator is a tridentate ligand.

8. The compound of claim 7, wherein the iron chelator inhibits proliferation of the cell via coordination of the donor atoms to iron, thereby sequestering iron.

9. The compound of claim 1, wherein the carbohydrate moiety comprises a glucose molecule, a mannose molecule, or a sugar derivative.

10. The compound of claim 1, wherein the carbohydrate moiety enables uptake of the compound into a cell via a glucose transporter protein.

11. A method of inhibiting proliferation of a cancer cell, said method comprising introducing to the cancer cell a compound according to claim 1.

12. A method of treating a condition associated with a metal ion dysregulation in a subject in need of such treatment, said method comprising administering to the subject a therapeutically effective amount of a compound comprising a prochelator conjugated to a carbohydrate moiety.

13. The method of claim 12, wherein the prochelator comprises a disulfide bond and at least two donor atoms.

14. The method of claim 13, wherein the prochelator is linked to the carbohydrate moiety by the disulfide bond.

15. The method of claim 13, wherein the prochelator comprises any one of the following: 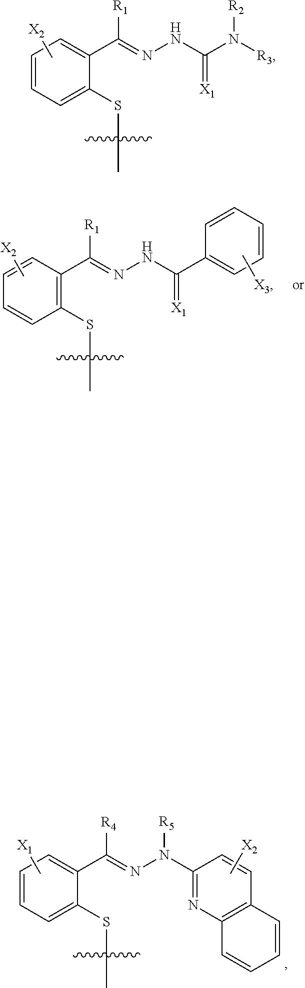
wherein R₁ is a Ph, pyridyl, p-CF₃-Ph, p-NO₂-Ph, CCl₃ or CF₃;
wherein R₂, R₃, R₄ are independently an H, alkyl, aryl, or substituted aryl;
wherein R₅ is an H or alkyl;
wherein X₁ is an O or S; and
wherein X₂ and X₃ are independently an H, alkyl, alkoxy, halide, CF₃, or NO₂.

16. The method of claim 12, wherein the carbohydrate moiety comprises a glucose molecule, a mannose molecule, or a sugar derivative.

17. A method of synthesizing a glycoconjugated compound, said method comprising:
a. initiating amidic or ester coupling of a pyridyl disulfide crosslinker with a carbohydrate moiety to produce a cross-linked carbohydrate; and
b. initiating disulfide exchange of the cross-linked carbohydrate with a chelator to link the chelator to the carbohydrate moiety via a disulfide bond, thereby producing the glycoconjugated compound.

18. The method of claim 16, wherein the pyridyl disulfide crosslinker is:

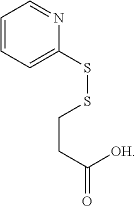 [SMILES: O=C(O)CCSSc1ccccn1]

19. The method of claim 16, wherein the chelator is according to any one of the following: 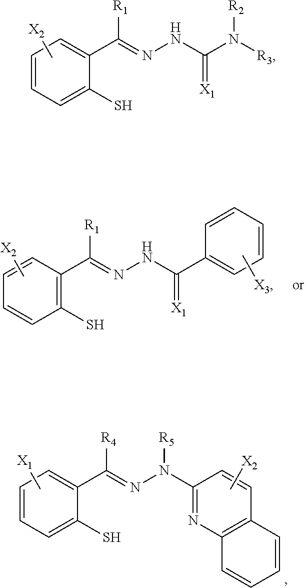
wherein R₁ is a Ph, pyridyl, p-CF₃-Ph, p-NO₂-Ph, CCl₃ or CF₃;
wherein R₂, R₃, R₄ are independently an H, alkyl, aryl, or substituted aryl;
wherein R₅ is an H or alkyl;
wherein X₁ is an O or S; and
wherein X₂ and X₃ are independently an H, alkyl, alkoxy, halide, CF₃, or NO₂.

20. The method of claim 16, wherein the carbohydrate moiety comprises a glucose molecule, a mannose molecule, or a sugar derivative.
